(19) United States
(12) Patent Application Publication
(10) Pub. No.: US 2018/0153952 A1
(43) Pub. Date: Jun. 7, 2018

(54) COMPOSITIONS AND METHODS FOR TREATING POST OPERATIVE COGNITIVE DYSFUNCTION AND NEUROINFLAMMATION WITH ANNEXIN A1 (ANXA1) PEPTIDES

(71) Applicant: Duke University, Durham, NC (US)

(72) Inventors: Zhiquan Zhang, Durham, NC (US); Niccolo Terrando, Durham, NC (US); Qing Ma, Durham, NC (US); Mihai V. Podgoreanu, Chapel Hill, NC (US); Joseph P. Mathew, Durham, NC (US); Mark F. Newman, Lexington, KY (US)

(21) Appl. No.: 15/829,387

(22) Filed: Dec. 1, 2017

Related U.S. Application Data
(60) Provisional application No. 62/429,247, filed on Dec. 2, 2016.

(51) Int. Cl. A61K 38/07; A61K 38/17

(52) U.S. Cl. CPC A61K 38/07; A61K 38/1709

(57) ABSTRACT

The present disclosure provides compositions and methods for treating post-operative cognitive dysfunction with Annexin A1 (ANXA1) peptides.

### PRIORITY STATEMENT

This application claims the benefit, under 35 U.S.C. § 119(e), of U.S. Provisional Application Ser. No. 62/429,247, filed Dec. 2, 2016, the entire contents of which are incorporated by reference herein.

### STATEMENT REGARDING ELECTRONIC FILING OF A SEQUENCE LISTING

A Sequence Listing in ASCII text format, submitted under 37 C.F.R. § 1.821, entitled 5405-500_ST25.txt, 16,854 bytes in size, generated on Dec. 1, 2017 and filed via EFS-Web, is provided in lieu of a paper copy. This Sequence Listing is incorporated by reference into the specification for its disclosures.

### FIELD OF THE INVENTION

This invention is directed to use of annexin A1 peptides for treatment, prevention, reduction and/or amelioration of postoperative cognitive dysfunction (POCD) and neuroinflammation.

### BACKGROUND

Surgery can be a life-saving procedure to restore function and enhance quality of life. However, neurologic complications after major surgery, including cardiac and orthopedic surgery, are common especially in a rapidly growing aging population. These complications include acute delirium and longer-lasting postoperative cognitive dysfunction (POCD), both of which are associated with higher mortality rates, decreased quality of life, and higher healthcare costs. We have established a clinically relevant mouse model of orthopedic surgery (commonly associated with postoperative cognitive complications) and linked surgical trauma to the development of inflammation in brain regions that are responsible for memory function (PNAS 2016; 113:E6686). Activation of the peripheral immune system leads to changes in blood-brain barrier, allowing macrophages to migrate into the brain parenchyma, and contributing to memory impairments. Although these mechanisms seem to apply to the human disease, the pathogenesis of these complications is complex and yet poorly understood.

Neuroinflammation and aging are the major factors for POCD. Annexin-A1 (ANXA1), an endogenous anti-inflammatory mediator (Nature 1979; 278:456), inflammation-ending/pro-resolving molecule (Science 2015; 347:6217), and NF-κB modulator (Cancer Res 2010; 70:2319) have been implicated in neuroprotection through resolution of inflammation, and recently shown to regulate histone deacetylases. However, it is not yet known whether ANXA1 regulates sirtuins (SIRTs), NAD⁺-dependent protein deacetylases, in conditions like memory impairments after surgery.

Sirtuins (SIRTs) are critical proteins involved in a wide range of cellular processes, including metabolism, inflammation, senescence, and overall lifespan or even healthspan. Seven members of the SIRT family (SIRT1-7, Table 1) have been identified in mammals. All share the same highly conserved NAD⁺-binding site and a Sir2 catalytic core domain with variable amino and carboxyl residues. SIRT1-3 and SIRT5-7 catalyze NAD⁺-dependent substrate-specific protein deacetylation, whereas SIRT4 acts as a NAD⁺-dependent mono-ADP-ribosyltransferase. SIRT6 has both deacetylase and auto-ADP-ribosyltransferase properties.

Among SIRTs (SIRT1-7), SIRT3 is unique because it is the only analogue that, with increased expression, has been correlated with extended lifespan and enhanced healthspan in humans. As shown in FIG. 1, SIRT3 regulates post-translational modification by removing the acetyl group (Ac) from a wide range of proteins involved in a variety of age-related diseases such as Alzheimer, neurodegenerative diseases, cancer, and cardiovascular diseases. SIRT3 is localized predominantly in the mitochondrial matrix and is referred to as a mitochondrial stress sensor that can modulate the activity of several mitochondrial proteins involved in metabolism, oxidative stress, fatty acid oxidation, and maintenance of cellular ATP levels. In addition, SIRT3 is required for recovery of mitochondria membrane potential (Cell 167:985, Nov. 3, 2016).

SIRT1 has been implicated in the prevention of many age-related diseases such as cancer, Alzheimer's disease, and type-2 diabetes. At the cellular level, SIRT1 controls DNA repair and apoptosis, circadian clocks, inflammatory pathways, insulin secretion, and mitochondrial biogenesis. Thus, increased expression of SIRTs—especially SIRT1, 3, and 6—by a small molecule activator (like our peptide), could be beneficial for patients at risk for cognitive dysfunction for example orthopedic surgery, cardiac surgery and transplantation, as well as other age-related complications and surgical procedures.

SIRT6 has deacetylase activity against histone substrates and weak ADP-ribosyltransferase activity in vitro (FIG. 2). SIRT6 can also remove long-chain acyl groups from peptides in vitro, which is more efficient than its deacetylase activity against peptides derived from H3K9. The long-chain deacylase activity for SIRT6 modulates tumor necrosis factor α (TNFα) by controlling its secretion rate. Overall, it appears that, through its effects on histone deacetylation, SIRT6 protects against aging and the diseases of aging. SIRT6 promotes genomic stability and helps to maintain telomere integrity. Remarkably, SIRT6 overexpression in male mice increased lifespan by ˜15%. In addition, SIRT6 protects against several age-related diseases, including cancer and diabetes. Thus, SIRT6 plays an important role in maintaining both lifespan and healthspan.

The inflammasome is a large multiprotein complex which plays a key role in innate immunity by participating in the production of the pro-inflammatory cytokines interleukin-1β (IL-1β) and IL-18. This requires activation of caspase-1, which occurs within the inflammasome following its assembly. The best characterized inflammasome is the NLRP3-NOD-like receptor family, pyrin domain containing 3 (FIG. 3). It comprises the NLR protein NLRP3, the adapter ASC and pro-caspase-1. The general consensus is that maturation and release of IL-1β requires two distinct signals: the first signal leads to synthesis of pro-IL-1β and other components of the inflammasome, such as NLRP3 itself; the second signal results in the assembly of the NLRP3 inflammasome, caspase-1 activation and IL-1β secretion. Activation of the NRLP3 inflammasome can be triggered by numerous stimuli, chemically and structurally highly different. Although the importance of the NLRP3 inflammasome in health and disease is well appreciated, a precise characterization of NLRP3 expression is yet undetermined.

### SUMMARY OF THE INVENTION

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

One aspect of the present disclosure provides a method for preventing or reducing cognitive decline in a subject following an inflammatory trigger generated by surgery, such as orthopedic, comprising, consisting of, or consisting essentially of administering to the subject a therapeutically effective amount of an ANXA1 peptide.

Another aspect of the present disclosure provides a method of treating cognitive decline in a subject following an inflammatory trigger comprising, consisting of, or consisting essentially of administering to the subject a therapeutically effective amount of an ANXA1 peptide.

Another embodiment of the present disclosure provides a method of ameliorating cognitive decline in a subject following an inflammatory trigger comprising, consisting of, or consisting essentially of administering to the subject a therapeutically effective amount of an ANXA1 peptide.

In another aspect, the present disclosure provides for the use of a therapeutically effective amount of an ANXA1 peptide in the manufacture of a medicament for use in preventing, reducing, treating, or ameliorating cognitive decline in a subject following an inflammatory trigger.

In some embodiments, the inflammatory trigger comprises an inflammatory trigger generated by a surgical procedure. In other embodiments, the trigger is a planned inflammatory trigger generated by a surgical procedure. In such embodiments, the cognitive decline comprises post-operative cognitive dysfunction (POCD).

In certain embodiments, the ANXA1 peptide comprises ANXA1sp.

Another aspect of the present disclosure provides all that is disclosed and illustrated herein.

Other objects and advantages will become apparent upon a review of the following description and figures.

### BRIEF DESCRIPTION OF THE DRAWINGS

The foregoing aspects and other features of the disclosure are explained in the following description, taken in connection with the accompanying drawings, herein:

FIG. 1 illustrates biological functions of a major mitochondrial NAD+-dependent protein deacetylase SIRT3.

FIG. 2 is a schematic summary of SIRT6 targets and regulators.

FIG. 3 is a cartoon showing assembly of the NLRP3 inflammasome, caspase-1 activation and IL-1β secretion.

FIG. 4A and FIG. 4B show the time-dependent decreased in brain (hippocampus) ANXA1 protein expression, which is accompanied by increased the plasma levels of meloperoxidase (MPO) in mice following orthopedic surgery.

FIG. 5 shows the time-dependent increased in brain NLRP expression/activation resulting in increased levels of activated caspase-1 and IL-113 secretion in the brain of mice following orthopedic surgery.

FIG. 6 shows the time-dependent increase in brain levels of acetylated NF-κB P65 (K310) subunit, which activates transcriptional activities for several downstream pro-inflammatory genes.

FIGS. 7A-7D show the time-dependent decreased in SIRT3 and increased in lysine (K122) acetylated superoxide dismutase 2 (Ac-SOD2, ↑ROS) in mice after orthopedic surgery.

FIG. 8 shows phosphorylated AMP-activated protein kinase α (pAMPKα, ↑ATP) via down-regulation of total liver kinase B1 (LKB1) in mice after orthopedic surgery.

FIG. 9 illustrates a novel effect of peripheral surgery on brain levels of OXPHOS mitochondrial electron transport chain complexes (mitochondria dynamic function) in mice following orthopedic surgery.

FIG. 10 illustrates a novel effect of peripheral surgery on brain levels of OXPHOS mitochondrial electron transport chain complexes (mitochondria dynamic function) with a significant reduction in mitochondrial numbers (citrate synthetase) in mice following orthopedic surgery.

FIG. 11 shows time-differential changes in several key players involved in pro-survival pathway, including SIRT6/H3K9 in mice following orthopedic surgery

FIG. 12 shows time-differential changes in several key players involved in pro-survival pathway, including FOXO3a in mice following orthopedic surgery.

FIG. 13 shows that mice treated with ANXA1sp significantly attenuated cerebral microglial activation in mice following orthopedic surgery compared to untreated animals.

FIG. 14 is showing that mice treated with ANXA1sp have significantly higher expression of mitochondrial SIRT3 in a time-dependent manner following orthopedic surgery.

FIG. 15A and FIG. 15B show that ANXA1sp significantly increased levels of hippocampal ANXA1, which was accompanied by significantly decreased plasma levels of meloperoxidase (MPO) in mice following orthopedic surgery.

FIGS. 16A-16C show that mice treated with ANXA1sp have significant inhibition of NLRP3 inflammasome activation induced by orthopedic surgery.

FIG. 17 shows the effects of ANXA-1sp on memory function after surgery.

FIGS. 18A-18F show age-dependent activation of NLRP3 inflammasome complex in the hippocampus after orthopedic surgery.

FIG. 19A and FIG. 19B show inflammatory changes in ASC^{-/-} mice.

FIG. 20 shows ASC-cit reporter mice display evident neuroinflammation.

FIG. 21A and FIG. 21B show ANXA1sp reduces NLRP3 activation.

FIGS. 22A-22D show the effects of ANXA1sp on other components of inflammasome complex.

FIGS. 23A-23F show time-differential changes in brain (hippocampus) OXPHOS complex (I-V) in mice after orthopedic surgery.

FIGS. 24A-24C show time-differential changes in brain citrate synthetase (CS, biomarker indicates numbers of mitochondria) in mice after orthopedic surgery.

FIGS. 25A-25C show orthopedic surgery impairs ANXA-1 in brain tissue.

FIGS. 26A-26C show systemic levels of IL-6 and MPO are reduced by ANXA-1sp.

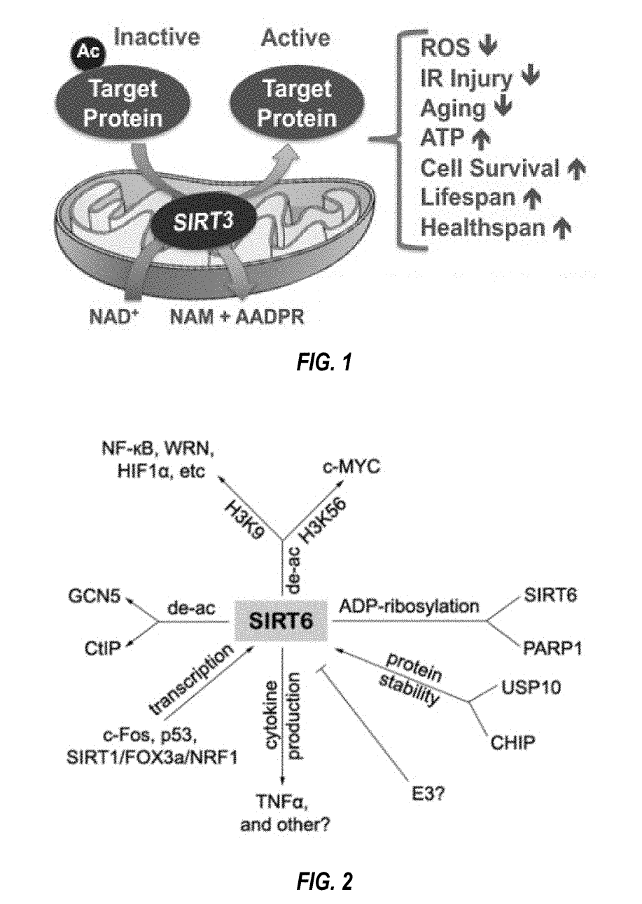

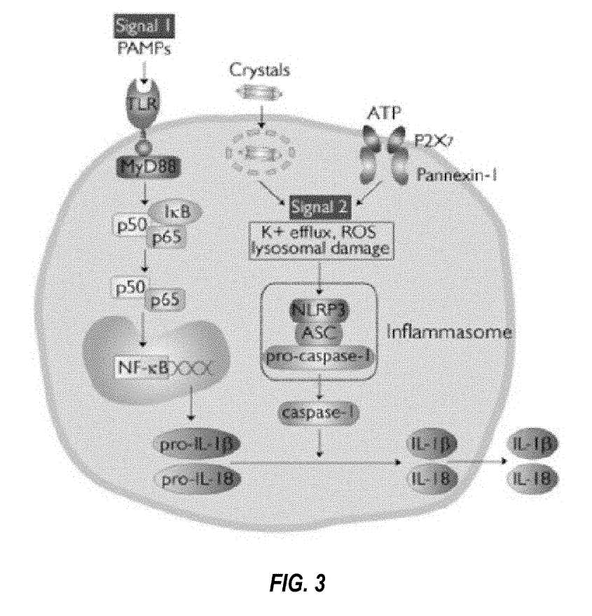

### DETAILED DESCRIPTION OF THE INVENTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates. The present invention will now be described more fully hereinafter with reference to the accompanying drawings and specification, in which preferred embodiments of the invention are shown. This invention may, however, be embodied in different forms and should not be construed as limited to the embodiments set forth herein.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Articles “a,” “an,” and “the” are used herein to refer to one or to more than one (i.e. at least one) of the grammatical object of the article. By way of example, “an element” means at least one element and can include more than one element.

As used herein, the term “and/or” refers to and encompasses any and all possible combinations of one or more of the associated listed items, as well as the lack of combinations when interpreted in an alternative (“or”).

“About” is used to provide flexibility to a numerical range endpoint by providing that a given value may be “slightly above” or “slightly below” the endpoint without affecting the desired result.

As used herein, the term “about,” when used in reference to a measurable value such as an amount of mass, dose, time, temperature, and the like, is meant to encompass variations of 20%, 10%, 5%, 1%, 0.5%, or even 0.1% of the specified amount.

As used herein, the term “one or more” or “one or more than one” can mean one, two, three, four, five, six, seven, eight, nine, ten or more, up to any number.

Unless otherwise defined, all technical terms used herein have the same meaning as commonly understood by one of ordinary skill in the art to which this disclosure belongs.

### Definitions

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition, such as post cognitive dysfunction following surgery or other inflammatory triggers. As used herein, the term “ameliorate” refers to the ability to make better or more tolerable, a condition, disorders and/or symptom. The term “prevent” refers to the ability to keep a condition, a reaction, a disorder and/or symptom from happening or existing or developing.

The term “effective amount” or “therapeutically effective amount” refers to an amount (e.g., of a compound such as ANXA 1) that is sufficient to effect beneficial or desirable biological and/or clinical results. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an effective amount or therapeutically effective amount in any individual case can be determined by one of ordinary skill in the art by reference to the pertinent texts and literature and/or by using routine experimentation. (See, for example, Remington, The Science and Practice of Pharmacy (latest edition)).

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. Preferably, the subject is a human patient that is suffering from cognitive decline following surgery or other inflammatory triggers.

A “subject in need thereof” or “a subject in need of” is a subject known to have, or is suspected of having, or developing, a cognitive decline following surgery or other inflammatory triggers.

The term “administering” or “administered” as used herein is meant to include topical, parenteral and/or oral administration, all of which are described herein. Parenteral administration includes, without limitation, intravenous, subcutaneous and/or intramuscular administration (e.g., skeletal muscle or cardiac muscle administration). In the methods of this present disclosure, the peptide of this present disclosure may be administered alone and/or simultaneously with one or more other compounds. In some embodiments, the compounds may be administered sequentially, in any order. It will be appreciated that the actual method and order of administration will vary according to, inter alia, the particular preparation of compound(s) being utilized, and the particular formulation(s) of the one or more other compounds being utilized. The optimal method and order of administration of the compounds of the present disclosure for a given set of conditions can be ascertained by those skilled in the art using conventional techniques and in view of the information set out herein.

The term “administering” or “administered” also refers, without limitation, to oral, sublingual, buccal, transnasal, transdermal, rectal, intramuscular, intravenous, intraarterial (intracoronary), intraventricular, intrathecal, and subcutaneous routes. In accordance with good clinical practice, the compounds of this present disclosure can be administered at a dose that will produce effective beneficial effects without causing undue harmful or untoward side effects, i.e., the benefits associated with administration outweigh the detrimental effects.

Human ANXA1 has a molecular weight of about 37 kDa and consists of about 346 amino acids. The amino acid sequence is coded for by nucleotides 75-1115 of the nucleotide sequence of GenBank® Accession number X05908 (SEQ ID NO:1) and is known by one skilled in the art as having the amino acid sequence of GenBank® Accession number P04083 (SEQ ID NO:2) (said sequences are incorporated by reference herein).

As used herein, the term “ANXA1 peptides” or “annexin A1 peptides” are peptide fragments of annexin 1, and are shorter than the full length ANXA1 protein (e.g., at least about 1, 2, 3, 4, 5, 6, 7, 8, 9, 10, 15, 20, 25, 30, 40, 50, 60, 70, 80, 90, 100, 125, 150, 175, 200, 225, 250, 275, 300, 325, 326, 327, 328, 329, 300, 331, 332, 333, 334, 335, 336, 337, 338, 339, 340, 341, 342, 434, 344, or 345 amino acids shorter), which have similar biological effects as ANXA1 on a cell, which biological activities are known in the art and as described herein. ANXA1 peptides may optionally be acetylated (Ac−) at the N-terminal amino acid residue. ANXA1 peptides include, but are not limited to, the ANXA1sp, Gln-Ala-Trp or Ac-Gln-Ala-Trp, the peptide Lys-Gln-Ala-Trp or Ac-Lys-Gln-Ala-Trp (SEQ ID NO:3); the peptide Phe-Leu-Lys or Ac-Phe-Leu-Lys, the peptide Phe-Gln-Ala-Trp or Ac-Phe-Gln-Ala-Trp (SEQ ID NO:4), the peptide Phe-Leu-Lys-Gln-Ala-Trp or Ac-Phe-Leu-Lys-Gln-Ala-Trp (SEQ ID NO:5), the peptide Glu-Phe-Leu-Lys-Gln-Ala-Trp (SEQ ID NO:6), the peptide Phe-Leu-Lys-Gln-Ala-Trp (SEQ ID NO:5), the peptide Ac-Ala-Met-Val-Ser-Glu-Phe-Leu-Lys-Gln-Ala-Trp or Ala-Met-Val-Ser-Glu-Phe-Leu-Lys-Gln-Ala-Trp (SEQ ID NO:7), the peptide Val-Ser-Glu-Phe-Leu-Lys-Gln-Ala-Trp or Ac-Val-Ser-Glu-Phe-Leu-Lys-Gln-Ala-Trp (SEQ ID NO:8) or other fragments of annexin 1 singly or in any combination, as long as they maintain the annexin 1 functionality. As used herein, the term “Ac2-26” refers to a 25mer peptide derived from annexin 1 having the sequence Ac-Ala-Met-Val-Ser-Glu-Phe-Leu-Lys-Gln-Ala-Trp-Phe-Ile-Glu-Asn-Glu-Glu-Gln-Glu-Tyr-Val-Gln-Tyr-Val-Lys (SEQ ID NO:9). As used herein, the term “ANXA1sp” or “annexin 1 short peptide” or “ANXA1 tripeptide” refers to the 3mer peptide derived from ANXA1 having the sequence: Ac-Gln-Ala-Trp.

An “inflammatory trigger” as used herein refers to any trigger that may lead to an inflammatory response in a subject that results, or may result, in a cognitive decline. By a “planned inflammatory trigger” we include the meaning of a planned medical procedure (e.g., a surgical procedure that occurs during surgery or a medical procedure that may occur in an in-patient or out-patient setting) that may be expected to lead to an inflammatory response in the patient, and where the planned inflammatory trigger has been associated with cognitive decline in patients. Thus, this may be any procedure that has been associated with post-procedure impaired cognition, which may be for example, delirium, dementia, confusion, as defined below.

By “cognitive decline” we include the meaning of any deterioration of cognitive function brought about by a cognitive disorder and/or an inflammatory trigger as defined below.

By “post-operative cognitive dysfunction”, we include the deterioration of intellectual function reflected as memory and concentration impairment presenting in a patient after that patient has undergone a surgical procedure. Such deterioration of intellectual function may take many forms and as such this definition includes any form of cognitive decline presenting post-operatively. The present disclosure is considered to be particularly useful when administered before, during or immediately following surgery. In general, cognitive dysfunctions following surgery are common and effective immediately following recovery. Classical POCD characterizes a more prolonged and subtle dysfunction in cognitive domains, juxtaposed to a more evident but short-lived “delirium” (both are included in the above definition of POCD). Discrimination between cognitive dysfunctions is made in particular according to the length of the cognitive impairment; delirium resolves itself usually after few days, whereas POCD persists for months (>3) and can become a permanent dysfunction. Thus, such cognitive decline falling within the scope of the above definition may be short-lived, thus may ablate hours or days after completion of the surgical procedure; or the cognitive decline may persist over the course of months or years, or the cognitive decline may even be permanent. Delirium is commonly seen after surgery, usually soon after surgery (hours to days) and fluctuating over time. Although the dysfunction lasts over a short period of time, delirium is associated with increased mortality (Ely et al. 2004), greater care dependency, costs (Milbrandt et al. 2004) and prolonged hospitalization (Ely et al. 2001). It is considered that the use of the present disclosure will aid in reducing or preventing this deterioration of intellectual function and lead to an improvement in the quality of life of the patient and his/her careers.

The diagnosis of POCD may be aided by neuropsychological testing. In general, the presence of POCD may be suspected when memory loss is greater than expected under normal situations. At present, there are no specific cognitive sets for successful POCD diagnosis; generally multiple neurocognitive assessments are made before reaching a diagnosis (Newman et al. Anesthesiology 106(3):572-90 (2007)).

It is envisaged that the symptoms of POCD may include memory loss, memory impairment, concentration impairment, delirium, dementia, and/or sickness behavior.

By “delirium” is included an acute and debilitating decline in attention, focus, perception, and cognition that produces an altered form of semi-consciousness. Delirium is a syndrome, or group of symptoms, caused by a disturbance in the normal functioning of the brain. The delirious patient has a reduced awareness of and responsiveness to the environment, which may be manifested as disorientation, incoherence, and memory disturbance. Delirium affects at least one in 10 hospitalized patients, and 1 in 2 elderly hospitalized patients. Whilst it is not a specific disease itself, patients with delirium usually fare worse than those with the same illness who do not have delirium. It occurs as a post-operative complication, with evidence from the mouse model described in the Examples showing that it can be caused by an inflammatory trigger. This would also explain why delirium is seen in patients admitted to hospital as a result of other inflammatory triggers, for example, stroke (CVA), Heart Attack (MI), urinary tract infection (UTI), respiratory tract infection (RTI), poisoning, alcohol or other medication withdrawal, hypoxia, and head injury.

By “dementia” we mean a serious cognitive disorder, which may be static, the result of a unique global brain injury or progressive, resulting in long-term decline in cognitive function due to damage or disease in the body beyond what might be expected from normal aging.

By “sickness behavior” are included symptoms ranging from lethargy, fever, decreased food intake, somnolence, hyperalgesia, and general fatigue to social withdrawal and memory impairment (Dantzer “Cytokine-induced sickness behaviour: a neuroimmune response to activation of innate immunity” Eur J Pharmacol 500(1-3):399-411 (2004)).

### Methods

The present disclosure provides, in part, compositions and methods for preventing, treating, ameliorating, reducing or inhibiting cognitive decline in patients following surgery, including orthopedic surgery, or other planned inflammatory triggers by administering to the subject an effective amount of an ANXA1 peptide.

One aspect of the present disclosure provides a method for preventing or reducing cognitive decline in a subject following an inflammatory trigger comprising, consisting of, or consisting essentially of administering to the subject a therapeutically effective amount of an ANXA1 peptide.

Another aspect of the present disclosure provides a method of treating cognitive decline in a subject following an inflammatory trigger comprising, consisting of, or consisting essentially of administering to the subject a therapeutically effective amount of an ANXA1 peptide.

Another embodiment of the present disclosure provides a method of ameliorating cognitive decline in a subject following an inflammatory trigger comprising, consisting of, or consisting essentially of administering to the subject a therapeutically effective amount of an ANXA1 peptide.

In another aspect, the present disclosure provides for the use of a therapeutically effective amount of an ANXA1 peptide in the manufacture of a medicament for use in preventing, reducing, treating, or ameliorating cognitive decline in a subject following an inflammatory trigger.

In some embodiments, the inflammatory trigger comprises an inflammatory trigger generated by a surgical or medical procedure. In other embodiments, the inflammatory trigger is a planned inflammatory trigger generated by a surgical or medical procedure. In such embodiments, the cognitive decline comprises post-operative cognitive dysfunction (POCD).

In certain embodiments, the ANXA1 peptide comprises ANXA1sp.

### Pharmaceutical Compositions

In addition to the ANXA1 peptides provided herein, pharmaceutical compositions of the present disclosure may contain one or more excipients or adjuvants. Selection of excipients and/or adjuvants and the amounts to use may be readily determined by the formulation scientist upon experience and consideration of standard procedures and reference works in the field.

Excipients such as diluents increase the bulk of a solid pharmaceutical composition, and may make a pharmaceutical dosage form containing the composition easier for the patient and care giver to handle. Diluents for solid compositions include, but are not limited to, microcrystalline cellulose (e.g., AVICEL®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT®), potassium chloride, powdered cellulose, sodium chloride, sorbitol, or talc.

Solid pharmaceutical compositions that are compacted into a dosage form, such as a tablet, may include, but are not limited to, excipients whose functions include, but are not limited to, helping to bind the active ingredient and other excipients together after compression, such as binders. Binders for solid pharmaceutical compositions include, but are not limited to, acacia, alginic acid, carbomer (e.g., CARBOPOL®), carboxymethylcellulose sodium, dextrin, ethyl cellulose, gelatin, guar gum, hydrogenated vegetable oil, hydroxyethyl cellulose, hydroxypropyl cellulose (e.g., KLUCEL®), hydroxypropyl methyl cellulose (e.g., METHOCEL®), liquid glucose, magnesium aluminum silicate, maltodextrin, methylcellulose, polymethacrylates, povidone (e.g., KOLLIDON®, PLASDONE®), pregelatinized starch, sodium alginate, or starch.

The dissolution rate of a compacted solid pharmaceutical composition in the subject's stomach may be increased by the addition of a disintegrant to the composition. Excipients which function as disintegrants include, but are not limited to, alginic acid, carboxymethylcellulose calcium, carboxymethylcellulose sodium (e.g., AC-DI-SOL®, PRIMELLOSE®), colloidal silicon dioxide, croscarmellose sodium, crospovidone (e.g., KOLLIDON®, POLYPLASDONE®), guar gum, magnesium aluminum silicate, methyl cellulose, microcrystalline cellulose, polacrilin potassium, powdered cellulose, pregelatinized starch, sodium alginate, sodium starch glycolate (e.g., EXPLOTAB®), or starch.

Glidants can be added to improve the flowability of a non-compacted solid composition and to improve the accuracy of dosing. Excipients that may function as glidants include, but are not limited to, colloidal silicon dioxide, magnesium trisilicate, powdered cellulose, starch, talc, or tribasic calcium phosphate.

When a dosage form such as a tablet is made by the compaction of a powdered composition, the composition is subjected to pressure from a punch and die. Some excipients and active ingredients have a tendency to adhere to the surfaces of the punch and die, which can cause the product to have pitting and other surface irregularities. A lubricant can be added to the composition to reduce adhesion and ease the release of the product from the die. Excipients that function as lubricants include, but are not limited to, magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, or zinc stearate.

Flavoring agents and flavor enhancers make the dosage form more palatable to the patient. Common flavoring agents and flavor enhancers for pharmaceutical products that may be included in the composition of the present disclosure include, but are not limited to, maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In liquid pharmaceutical compositions of the present disclosure, the active ingredient and any other solid excipients are suspended in a liquid carrier such as water, vegetable oil, alcohol, polyethylene glycol, propylene glycol, or glycerin. As used herein, “active ingredient” means ANXA1 peptides described herein.

Liquid pharmaceutical compositions may contain emulsifying agents to disperse uniformly throughout the composition an active ingredient or other excipient that is not soluble in the liquid carrier. Emulsifying agents that may be useful in liquid compositions of the present disclosure include, but are not limited to, gelatin, egg yolk, casein, cholesterol, acacia, tragacanth, chondrus, pectin, methyl cellulose, carbomer, cetostearyl alcohol, or cetyl alcohol.

Liquid pharmaceutical compositions of the present disclosure may also contain a viscosity enhancing agent to improve the mouth-feel of the product and/or coat the lining of the gastrointestinal tract. Such agents include, but are not limited to, acacia, alginic acid, bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, or xanthan gum.

Sweetening agents such as sorbitol, saccharin, sodium saccharin, sucrose, aspartame, fructose, mannitol, or invert sugar may be added to improve the taste.

Preservatives and chelating agents such as alcohol, sodium benzoate, butylated hydroxy toluene, butylated hydroxyanisole, or ethylenediamine tetraacetic acid may be added at levels safe for ingestion to improve storage stability.

According to the present disclosure, a liquid composition may also contain a buffer such as gluconic acid, lactic acid, citric acid or acetic acid, sodium gluconate, sodium lactate, sodium citrate, or sodium acetate.

An amount of an ANXA1 peptide adequate to accomplish therapeutic or prophylactic treatment as described herein is defined as a therapeutically or prophylactically-effective amount or as an effective amount. In both prophylactic and therapeutic regimens, ANXA1 peptides of the present disclosure can be administered in several dosages until a desired effect has been achieved.

Effective doses of the compositions of the present disclosure, for the treatment of the above described conditions vary depending upon many different factors, including means or mode of administration, target site, physiological state of the subject, whether the subject is human or an animal, other medications administered, and whether treatment is prophylactic or therapeutic. Usually, the subject is a human but nonhuman mammals including transgenic mammals can also be treated. Treatment dosages can be titrated to optimize safety and efficacy. Generally, an effective amount of the agents described above will be determined by the age, weight and condition or severity of disease of the subject.

The amount of ANXA1 peptide depends on whether additional active and/or inactive compounds, such as pharmaceutical carriers, are also administered, with higher dosages being required in the absence of additional compounds. The amount of an ANXA1 peptide for administration can be from about 1 mg to about 500 μg per patient and in some embodiments can be from about 5 μg to about 500 mg per administration for human administration. In particular embodiments, a higher dose of about 1-2 mg per administration can be used. Typically about 5, 10, 20, 50 or 100 μg is used for each human administration.

Generally, dosing may be one or more times daily, or less frequently, such as once a day, once a week, once a month, once a year, once in a decade, etc. and may be in conjunction with other compositions as described herein. In certain embodiments, the dosage is greater than about 1 μg/subject and usually greater than about 10 μg/subject if additional compounds are also administered, and greater than about 10 μg/subject and usually greater than about 100 μg/subject in the absence of additional compounds, such as a pharmaceutical carrier. It should be noted that the present disclosure is not limited to the dosages recited herein.

The dosage and frequency of administration can vary depending on whether the treatment is prophylactic or therapeutic. In prophylactic applications, a relatively low dosage can be administered at relatively infrequent intervals over a long period of time. Some patients may continue to receive treatment for the rest of their lives. In therapeutic applications, a relatively high dosage at relatively short intervals is sometimes required until severity of the injury is reduced or terminated, and preferably until the subject shows partial or complete amelioration of symptoms of injury. Thereafter, the subject can be administered a prophylactic regimen.

The aforementioned embodiments are not exclusive and may be combined in whole or in part. As will be understood by one skilled in the art, there are several embodiments and elements for each aspect of the claimed disclosure, and all combinations of different elements are hereby anticipated, so the specific combinations exemplified herein are not to be construed as limitations in the scope of the disclosure as claimed. If specific elements are removed or added to the group of elements available in a combination, then the group of elements is to be construed as having incorporated such a change.

Another aspect of the present disclosure provides all that is disclosed and illustrated herein.

The present subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### EXAMPLES

The following EXAMPLES provide illustrative embodiments. Certain aspects of the following EXAMPLES are disclosed in terms of techniques and procedures found or contemplated by the present inventors to work well in the practice of the embodiments. In light of the present disclosure and the general level of skill in the art, those of skill will appreciate that the following EXAMPLES are intended to be exemplary only and that numerous changes, modifications, and alterations can be employed without departing from the scope of the presently claimed subject matter.

### Example 1: Novel Signaling Pathways in Surgery-Induced Cognitive Dysfunction

We have recently identified time-differential changes in several endogenous proteins that involve in ANXA1-mediated pro-resolving mechanisms, SIRT-mediated pro-survival pathways, and NLRP3 inflammasome-activated inflammatory pathways in mouse brain (e.g. hippocampus, which is critically involved in memory function) after orthopedic surgery. Our data, for the first time, implicate these proteins, including ANXA1/MPO (neutrophil transmigration), NLRP3/Caspase-1/IL-1β (inflammasome assembly/activation), SIRT1/NF-kB (inflammation), SIRT3/LKB1/pAMPK/SOD2 (ATP, ROS), SIRT3/OXPHOS/CS (metabolism, mitochondria dynamics function) SIRT6/H3H9/FOXO3a (pro-survival mechanism) in a mouse model of surgery-induced cognitive dysfunction.

ANXA1/MPO—Neutrophil Transmigration-Inflammation.

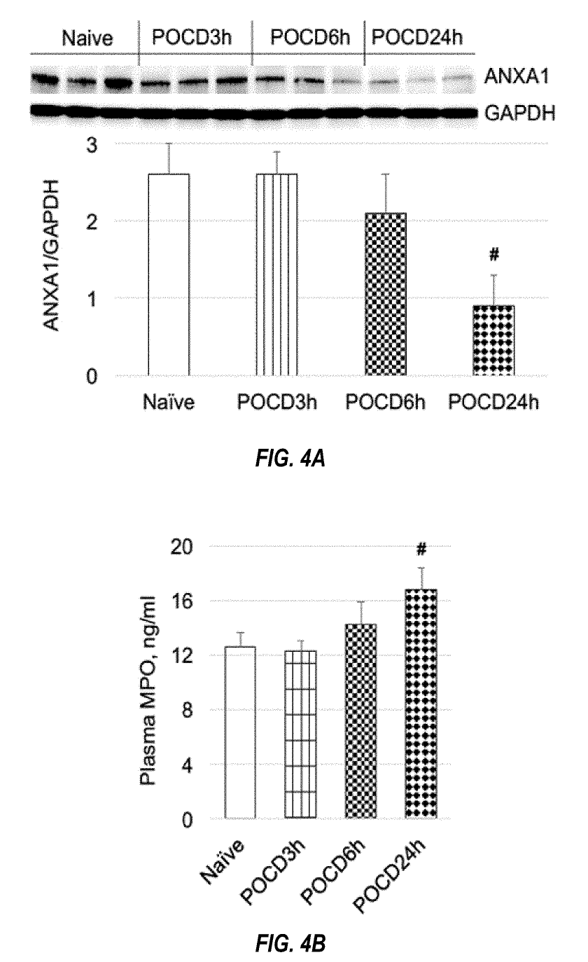

We have recently found time-dependent decreased in brain (hippocampus) ANXA1 protein expression (FIG. 4A), which is accompanied by increased the plasma levels of meloperoxidase (MPO) (FIG. 4B) in mice following orthopedic surgery.

NLRP3/Caspase-1/IL-1β—Inflammasome Assembly and Activation.

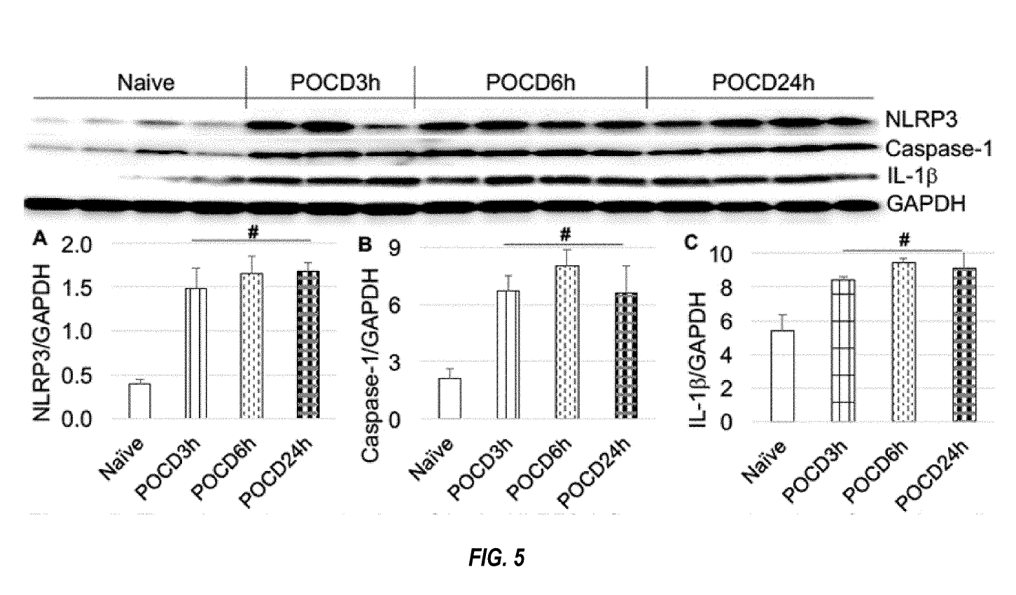

We have also found that time-dependent increased in brain NLRPs expression resulted in increased in caspase-1 and IL-1β in mice following orthopedic surgery (FIG. 5).

SIRT1/NF-kB—Inflammation-Transcriptional Activity.

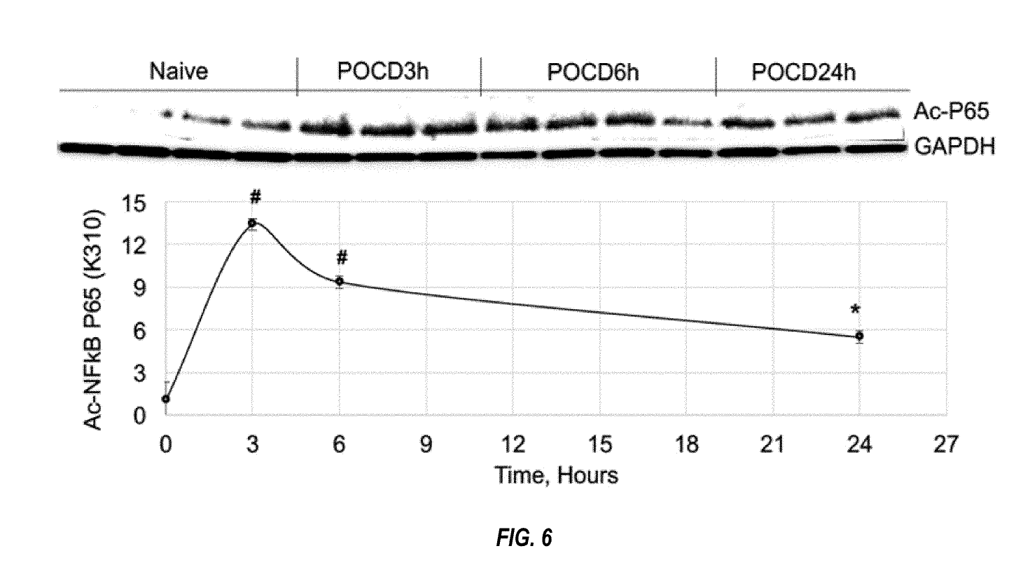

Furthermore, we found that the time-dependent suppression of SIRT1 expression resulted in increased the brain levels of acetylated NF-κB P65 (K310) subunit, which activates transcriptional activities for the downstream pro-inflammatory genes (FIG. 6).

SIRT3/SOD2—ROS.

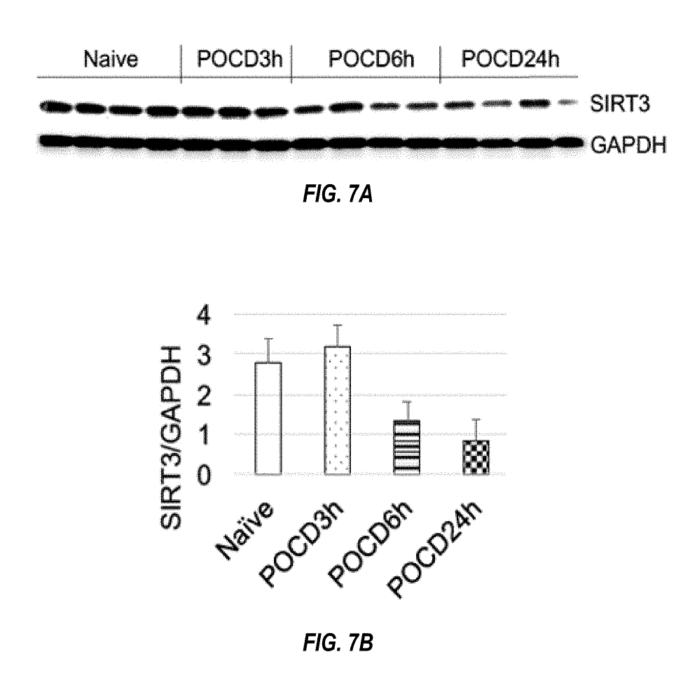

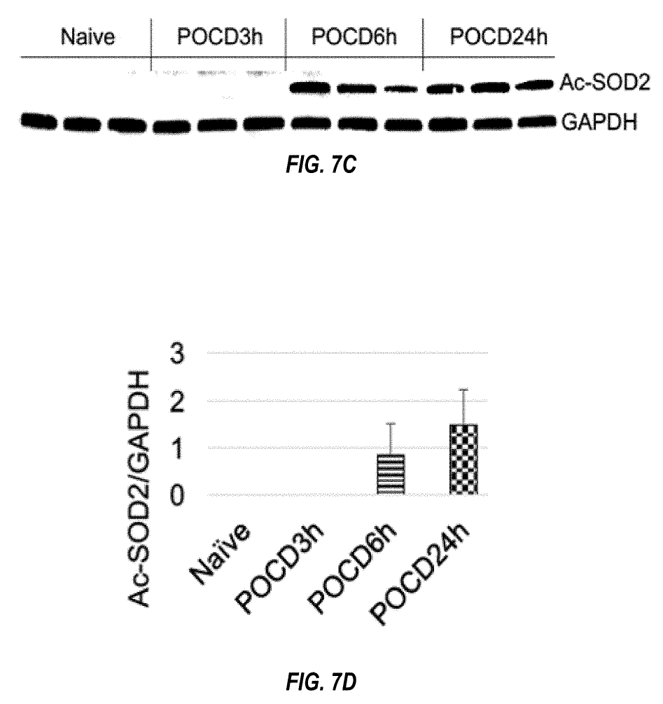

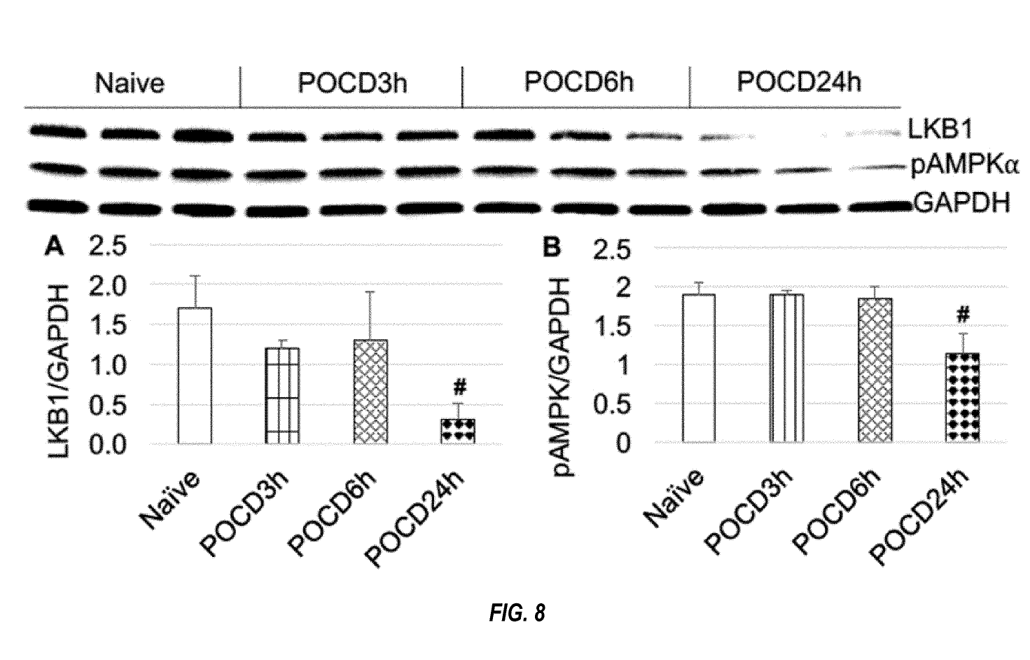

Moreover, we found the time-dependent decreased in SIRT3 and increased in lysine (K122) acetylated superoxide dismutase 2 (Ac-SOD2, ↑ROS) (FIG. 7) and phosphorylated AMP-activated protein kinase α (pAMPKα, ↑ATP) via down-regulation of total liver kinase B1 (LKB1) (FIG. 8), in mice after orthopedic surgery.

OXPHOS/CS—Mitochondria Dynamics Function.

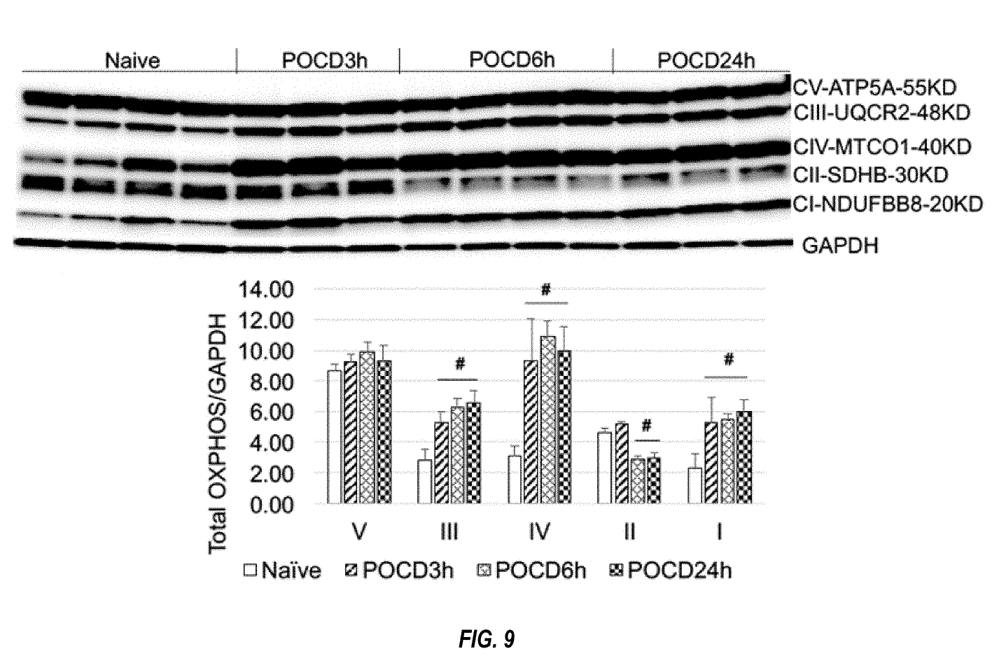

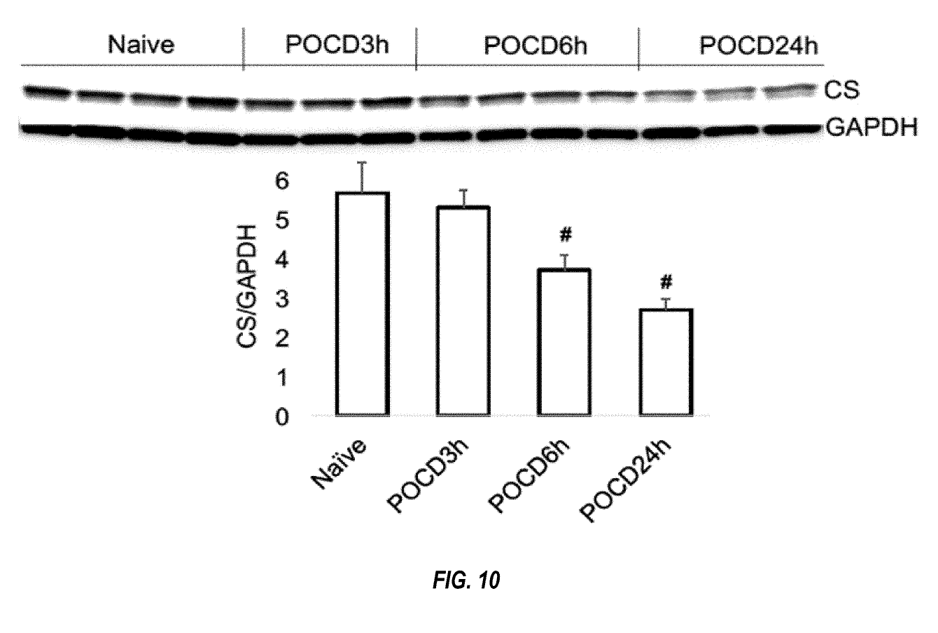

In addition, we have, for the first time, revealed a novel effect of peripheral surgery on brain levels of OXPHOS mitochondrial electron transport chain complexes (mitochondria dynamic function) (FIG. 9), with a significant reduction in mitochondrial numbers (citrate synthetase) (FIG. 10) in mice following orthopedic surgery.

SIRT6/H3K9/FOXO3a Pro-Survival Mechanism.

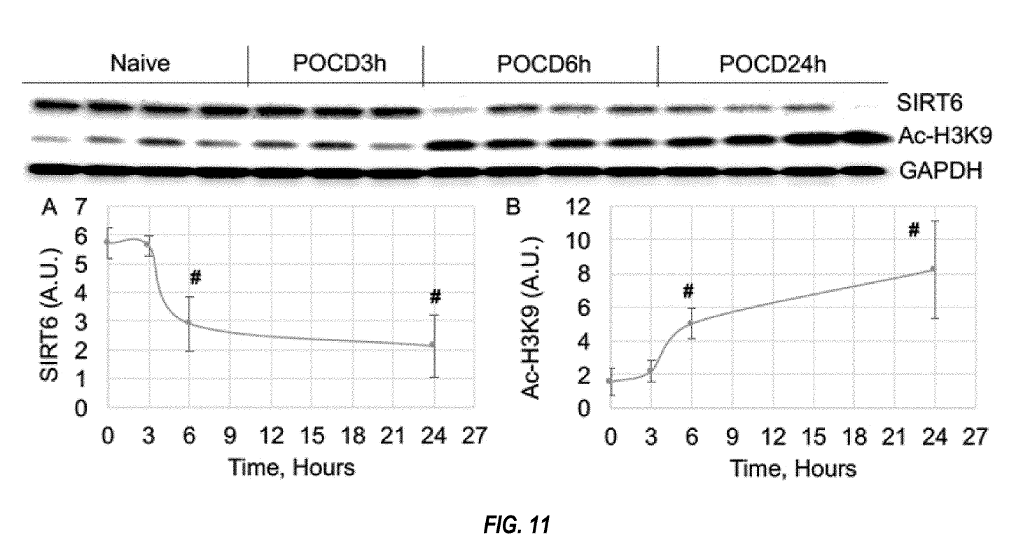

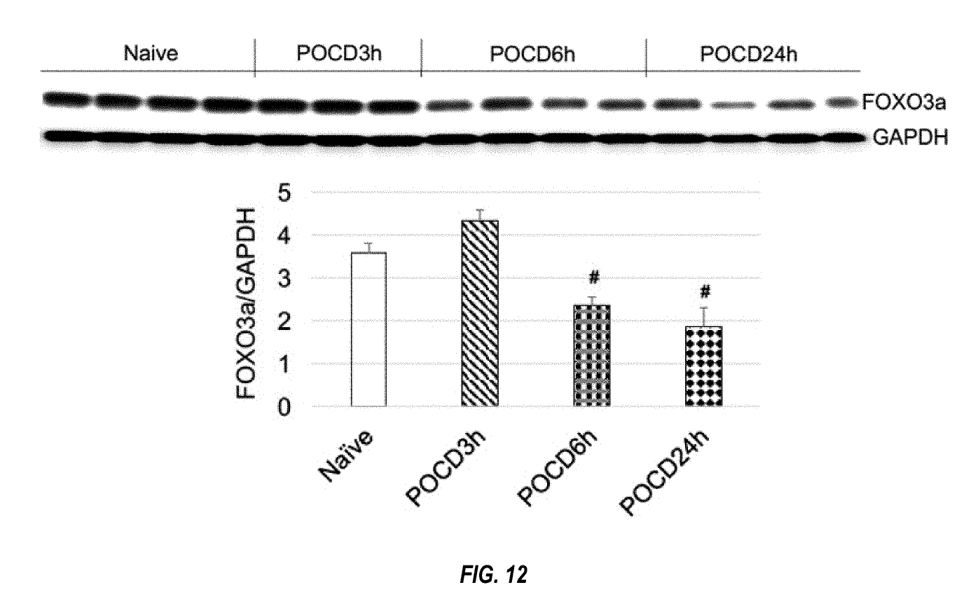

Finally, we have found time-differential changes in several key players involved in pro-survival pathway, including SIRT6/H3K9 (FIG. 11), and FOXO3a in mice following orthopedic surgery (FIG. 12).

### Example 2: ANXA1sp-Treatment for Surgery-Induced Cognitive Dysfunction

We have developed a novel tripeptide (ANXA1sp) derived from the N-terminal domain of human Annexin-A1 protein (ANXA1). Here we conduct studies to demonstrate that ANXA1sp protects the brain from POCD after orthopedic surgery through preventing/resolving cerebral inflammation and promoting pro-survival pathways.

ANXA1sp Attenuates Microglia Activation after Orthopedic Surgery.

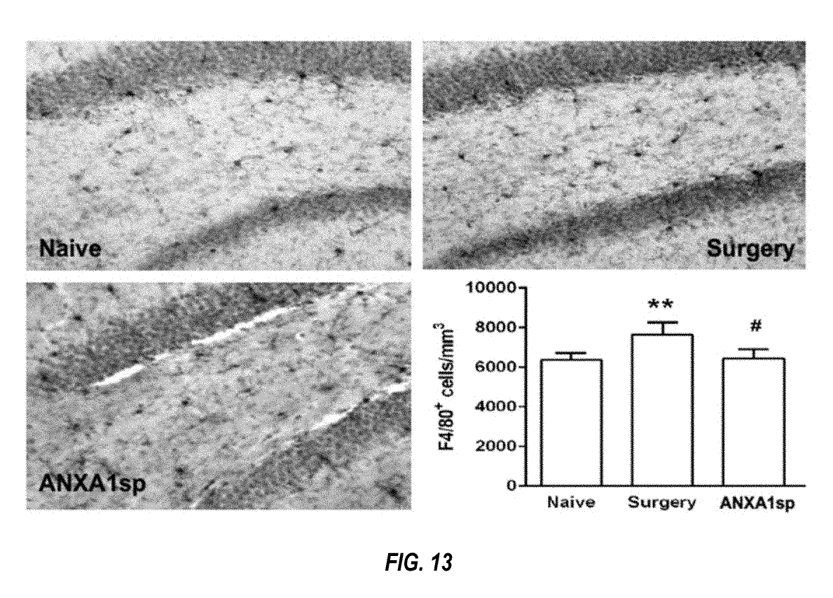

Our ongoing work has revealed mice treated with ANXA1sp significantly attenuated cerebral microglia activation in the brain of mice following orthopedic, compared to those untreated animals (FIG. 13).

ANXA1sp Increases/Activates Hippocampal SIRT3.

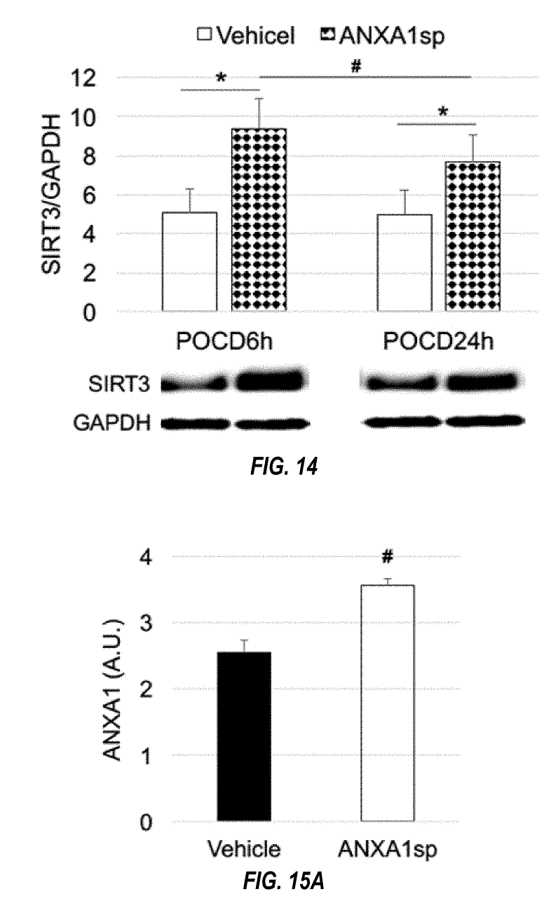

Furthermore, we have found that mice treated with ANXA1sp significantly increases/activates mitochondrial SIRT3 in a time-dependent manner following orthopedic surgery (FIG. 14).

ANXA1sp Attenuates Plasma Levels of MPO.

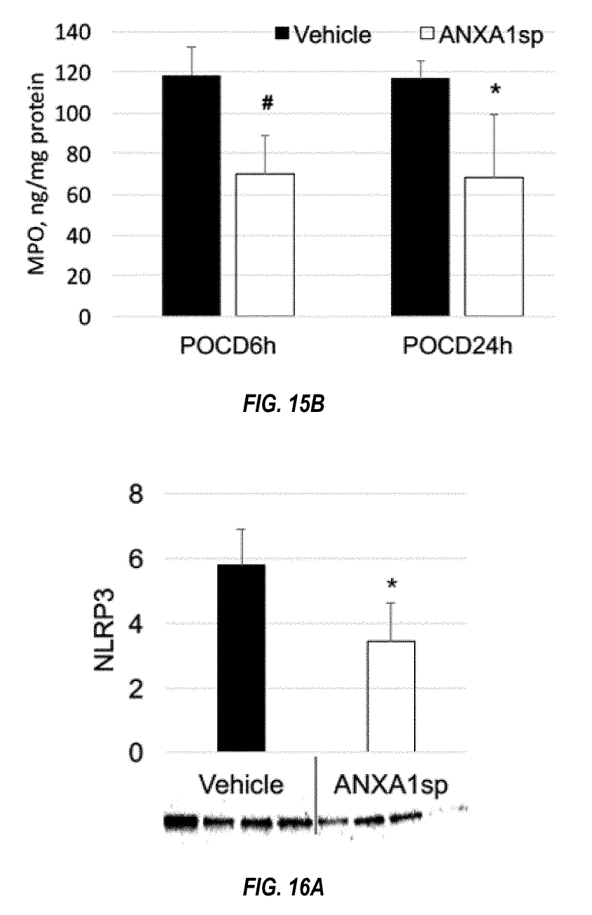

Moreover, we have found that mice treated with ANXA1sp significantly increased hippocampal ANXA1 and resulted in reduced plasma levels of MPO in a time-dependent manner after orthopedic surgery (FIG. 15).

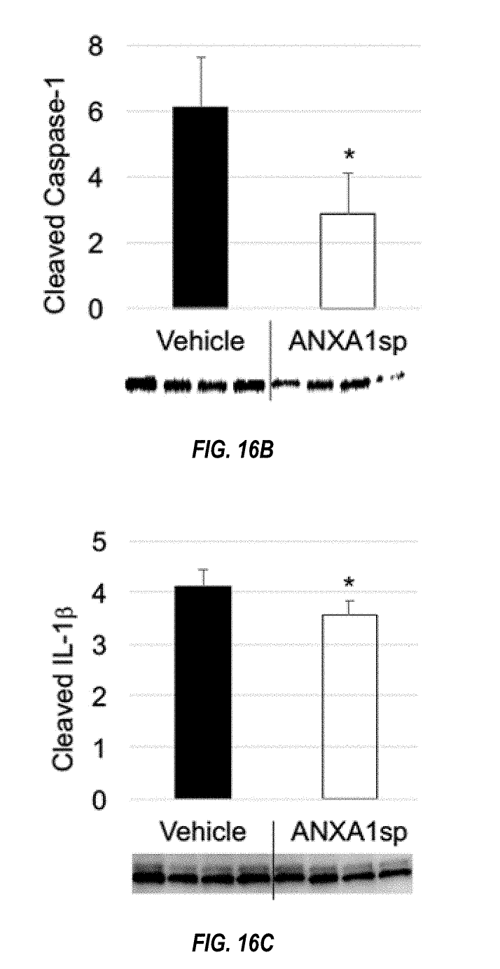

Finally, we have just revealed that ANXA1sp inhibits NLRP3 expression or NLRP3 inflammasome activation, which was accompanied by significantly inhibited activation of caspase-1 and IL-1β in the brain (hippocampus) of mice following orthopedic surgery (FIG. 16).

### Example 3: Additional Studies

Male C57BL/6 mice (12 weeks) were subjected to orthopedic surgery and treated with ANXA1sp (1 mg/kg) 1 hr before surgery. At 24 hr of post-surgery, brain levels of microglia activation was determined by immunostaining with a biomarker F4/80. Stereological analysis of F4/80 positive cells was shown in the bar graph. N=5 (Naïve), 10 (Surgery), and 4 (ANXA1sp). Data are presented as mean+SD. **P<0.01 vs. Naïve; #P<0.05 vs. Surgery. One-way ANOVA with Multiple Comparisons. (FIG. 13).

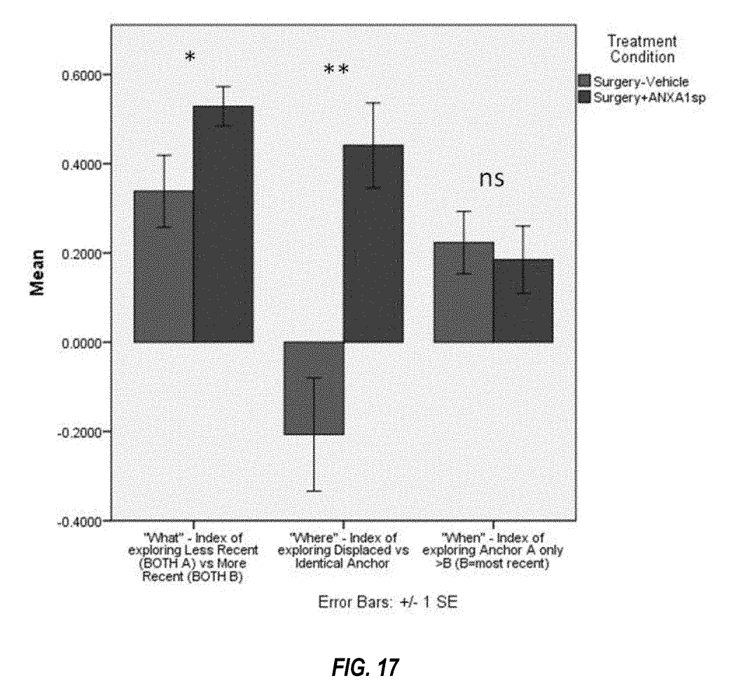

Results from the “what-when-where” task of object recognition memory, which provides a comparable assessment of human POCD. After orthopedic surgery mice have reduced preferences for the replacement of objects (what), and for the original location of the displaced object (where); but they show a weakened preference the order of presentation (when). This negative/weak preference score suggests that the mouse has memory deficits. Notably, treatment with ANXA-1sp significantly improves these deficits. In fact, surgery+ANXA1sp mice show a greater exploration of the displaced/replaced object with lower exploration/disinterest in the original anchor object and the two most recently seen objects, whereas the surgery+vehicle mice show no differences in any of these object presentation—indicating that they can't really discriminate and they don't recognize the displaced/replaced object vs the others. Data are presented as mean±SD. N=10. *P<0.05 and **P<0.01 vs. surgery+vehicle using MANOVA. (FIG. 17).

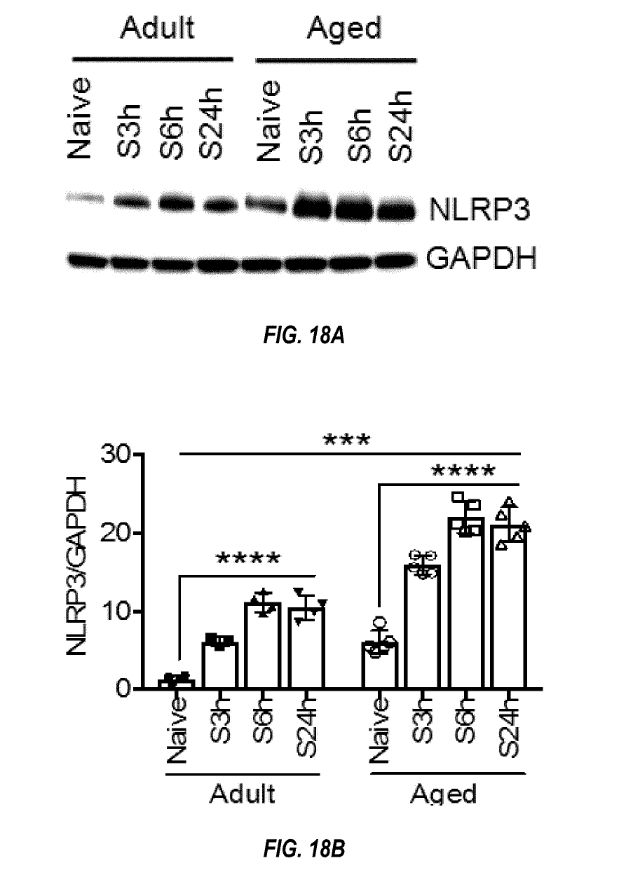

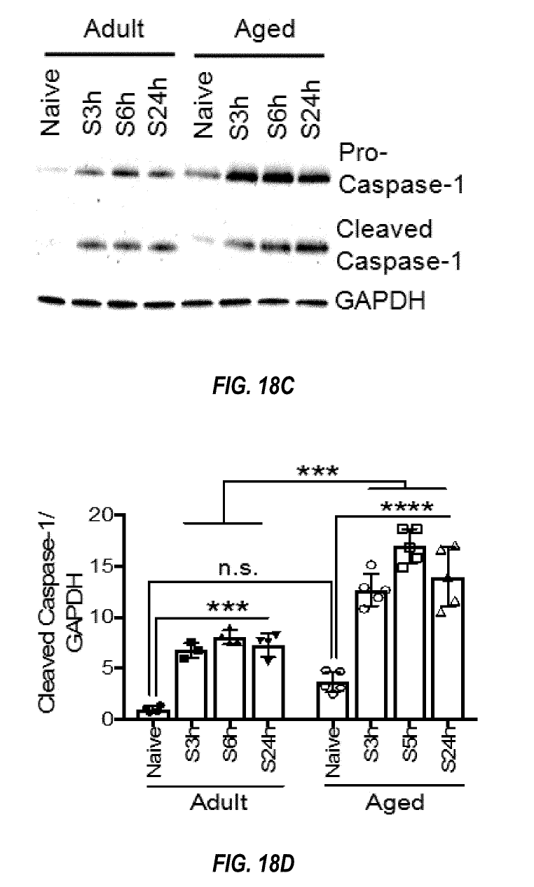

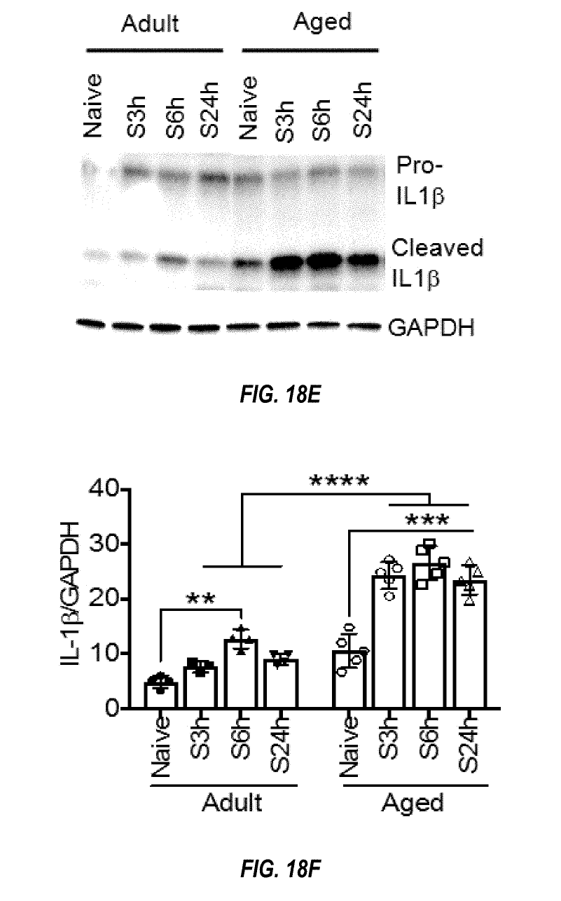

Representative Western Blot images in adult (12 weeks-old) and aged (20 months-old) C57BL/6 mice for NLRP3 (FIG. 18A), Caspase-1 (FIG. 18C), and IL1β (FIG. 18E) (three key components of the inflammasome complex) and quantification. Surgery induced a time-dependent activation of these makers starting at 3 hr and remaining significantly up-regulated up to 24 hr (FIG. 18B), (FIG. 18D), and (FIG. 18F). Aged mice showed further elevation of these markers. Data are presented as mean±SD. N=4 for adult, 5 for aged; **P<0.01, ***P<0.001, ****P<0.0001; n.s. no significance. One-way ANOVA with Multiple Comparisons.

To further ascertain the role of the NLPR-3 complex, we used knock-out mice and assessed microglia morphological changes. At 24 hr after orthopedic surgery, NLRP-3^{-/-} mice were protected from changes in microglia activation as measured by Iba-1 immunostaining.

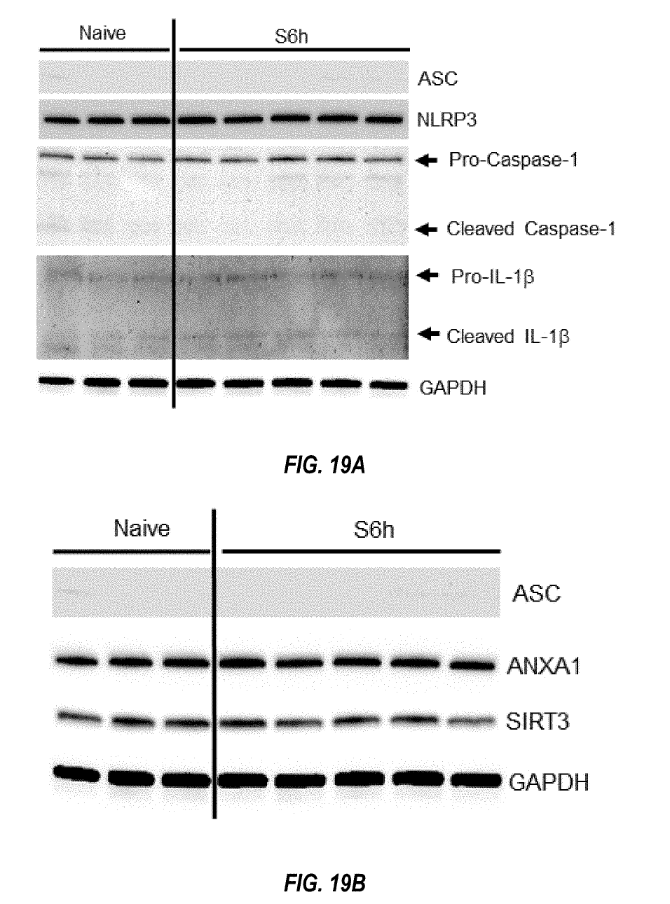

The inflammasome adaptor ASC is a cardinal for caspase-1 activation and IL1β secretion. We used mice lacking expression of ASC and found no changes in these downstream pathways, suggesting ASC is critical in the induction of inflammatory cytokines after surgery (FIG. 19A). Note that NLPR3 expression is maintained in these mice. Further, both ANXA-1 and SIRT-3 levels were sustained in the ASC^{-/-} mice, suggesting pro-resolving pathways are not affected (FIG. 19B). n=3 for Naïve, 5 for S6h.

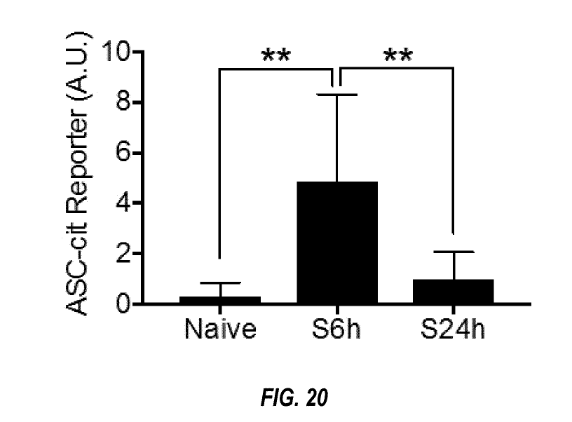

The ASC-citrine mouse is a transgenic model that reports inflammasome activation. Following surgery we observed significant “specks” formation at 6 hr and 24 hr (demonstrating assembly of the inflammasome complex). We assessed microglial activation in these mice and found significant morphological changes starting at 6 hr (FIG. 20). Data are presented as mean±SD. **P<0.01, n=3 for (Naïve and S6h) n=4 (for S24h). ANOVA with Multiple Comparisons.

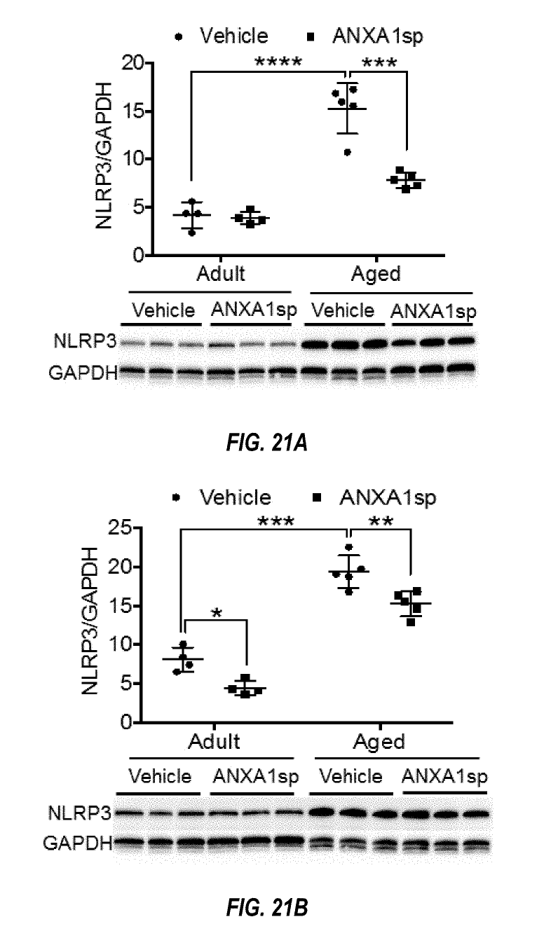

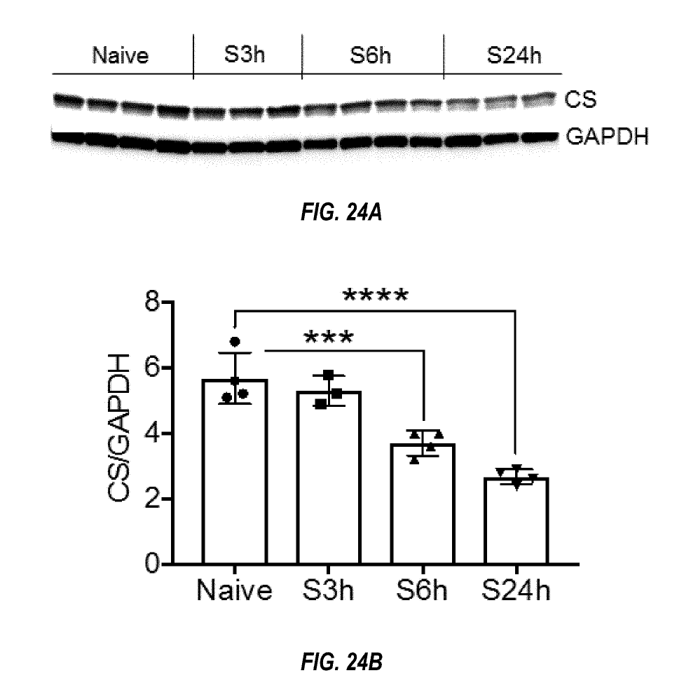

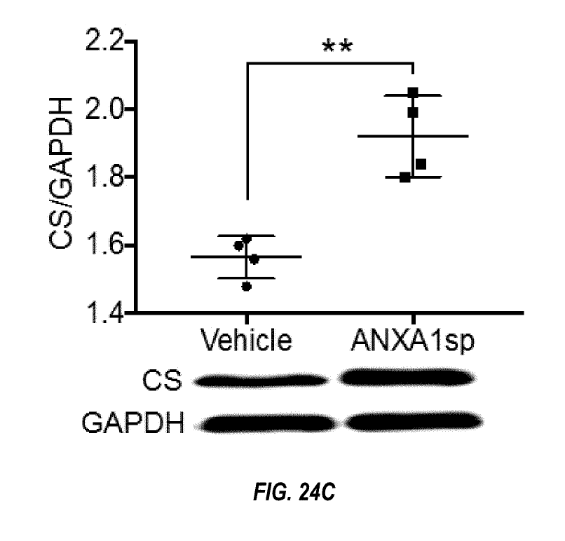

Male C57BL/6 mice received ANXA1sp (1 mg/kg) 30 min before surgery. ANXA1sp significantly reduced NLRP3 activation at 6 hr in aged mice after surgery (FIG. 21A) and 24 hr both in adult and aged groups (FIG. 21B). Data are presented as mean±SD. N=4-5; *P<0.05, **P<0.01, ***P<0.001; n.s. no significance. Two-way ANOVA with Multiple Comparisons.

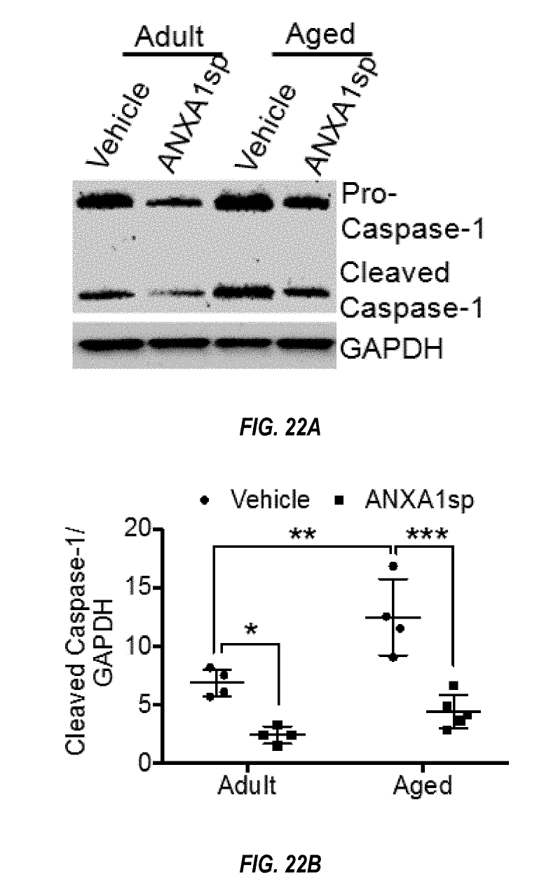

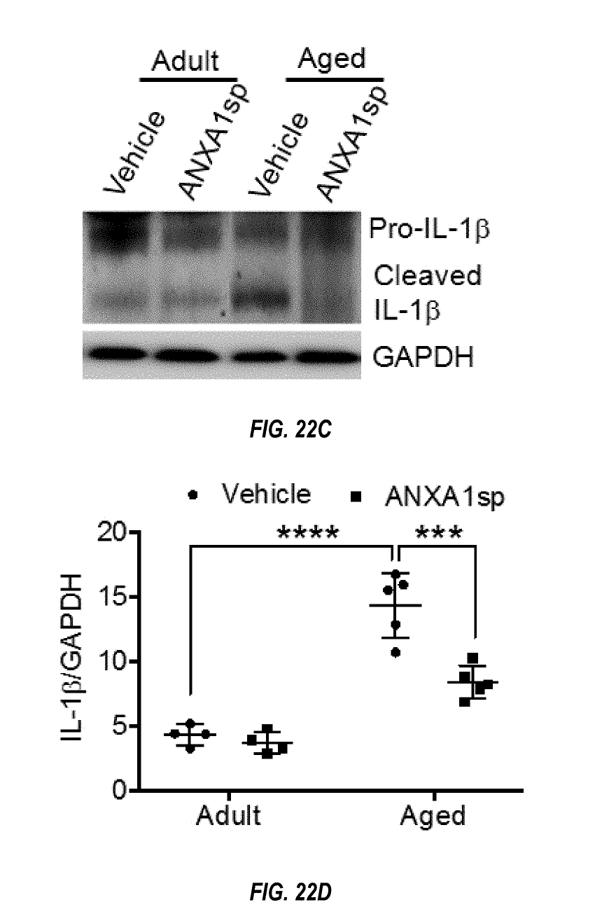

Male C57BL/6 mice received ANXA1sp (1 mg/kg) 30 min before surgery. Representative blots of Caspase-1 activation and IL1β secretion. ANXA 1sp significantly reduced levels of Caspase-1 (FIGS. 22A-B) in both adult and aged mice after surgery. Although no significance was reported in adult mice for secreted IL1β (FIGS. 22C-D), this was highly significant in aged mice. Data are presented as mean±SD. N=4-5; *P<0.05, **P<0.01, ***P<0.001. Two-way ANOVA with Multiple Comparisons.

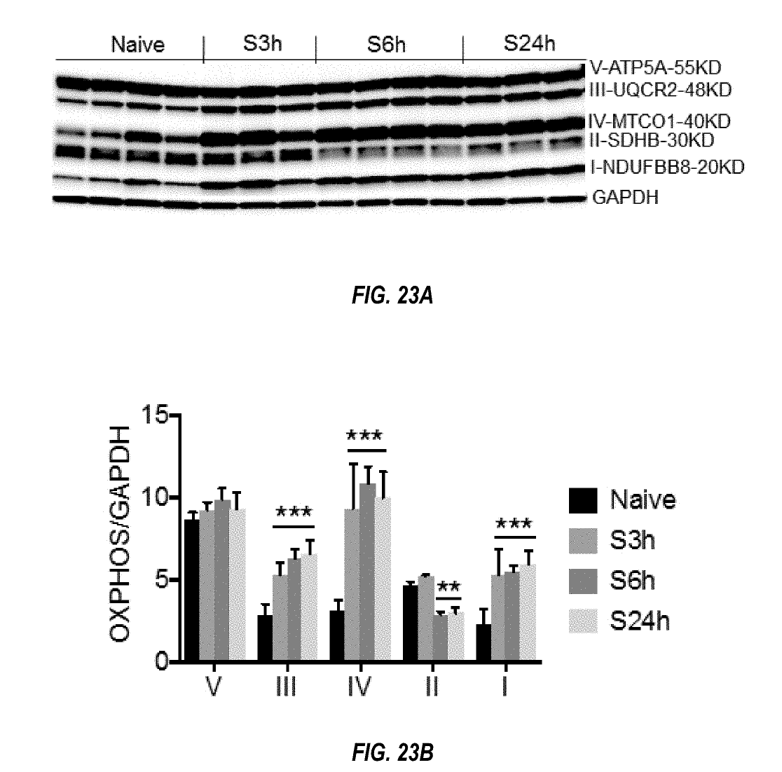

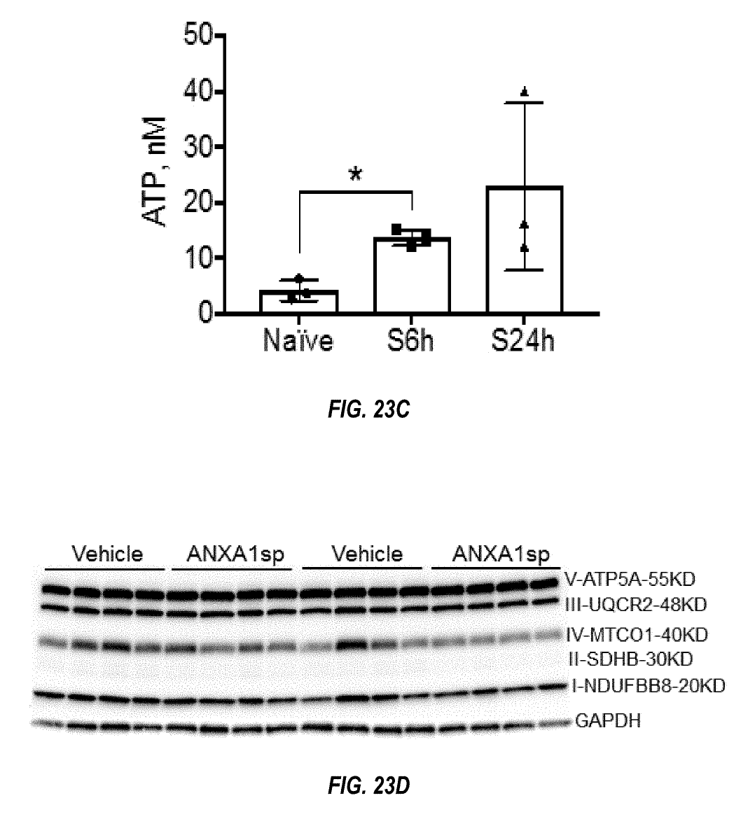

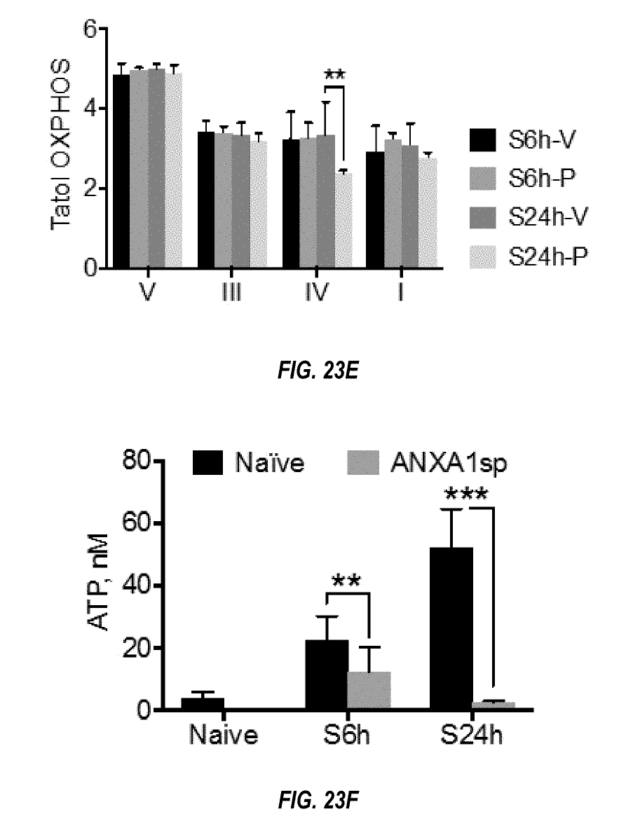

Male C57BL/6 mice (13 weeks) were subjected to orthopedic surgery. Naïve animals (no surgery at 0 hour) were used as baseline-control. At 3, 6, and 24 hours of post-surgery, the total OXPHOS in hippocampus was determined by Western blot (FIG. 23A) and (FIG. 23B). Time-dependent increased hippocampal ATP production were determined by ELISA (FIG. 23C). ANXA1sp significantly regulated hippocampal total OXPHOS, particularly in complex IV (FIG. 23D) and (FIG. 23E) and in turn attenuated hippocampal ATP production (FIG. 23F). Data are presented as mean±SD. N=4; **P<0.01, ***P<0.001. One-way ANOVA with Multiple Comparisons.

Male C57BL/6 mice (13 weeks) were subjected to orthopedic surgery while naïve animals were as baseline-control. At 3, 6, and 24 hours of post-surgery, the hippocampus CS was determined by Western blot (FIG. 24A) and (FIG. 24B). Animals treated with ANXA1sp significantly increased the numbers of CS that determined by Western blot (FIG. 24C). Data are presented as mean±SD. N=4; **P<0.01, ***P<0.001, ****P<0.0001. One-way ANOVA with Multiple Comparisons.

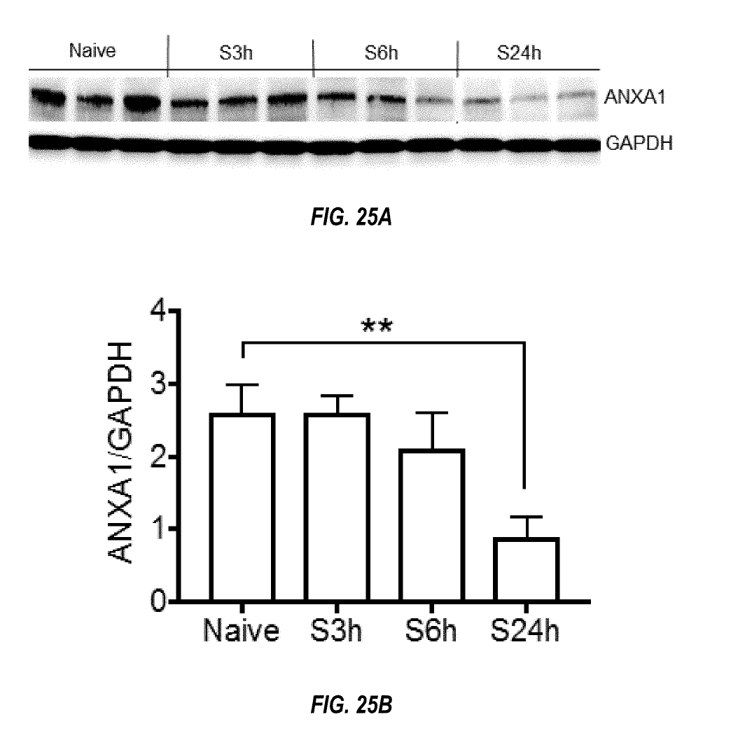

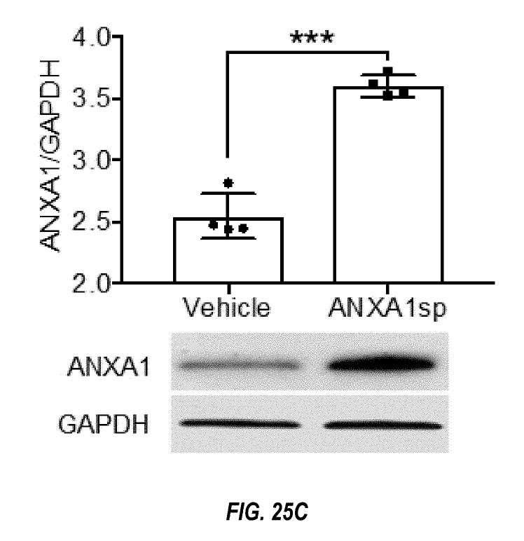

Surgery reduced levels of endogenous ANXA1 in the brain at 24 hr (FIG. 25A, FIG. 25B for quantification). Mice treated with 1 mg/kg of ANXA1sp significantly increase levels of ANXA-1 at 24 hr (FIG. 25C). N=3-4; **P<0.01, ***P<0.001. One-way ANOVA with Multiple Comparisons.

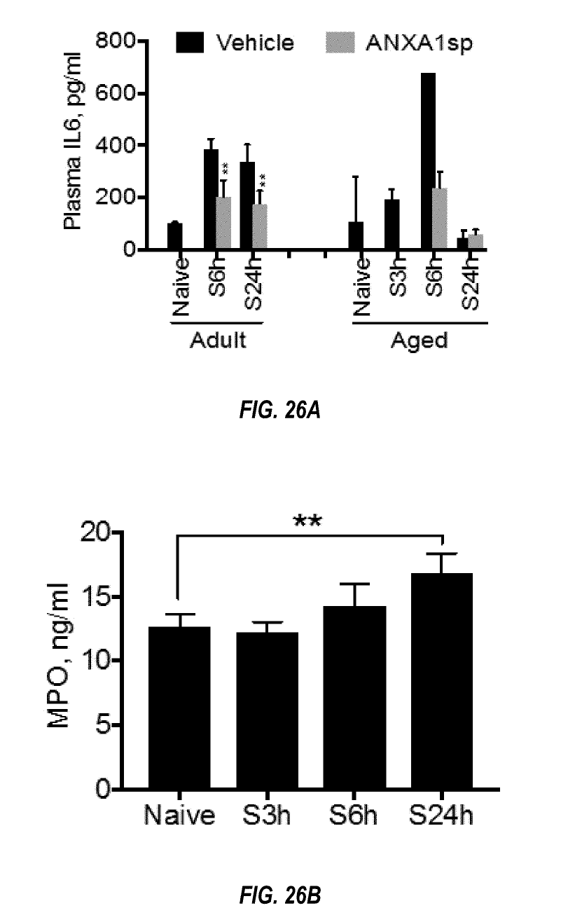

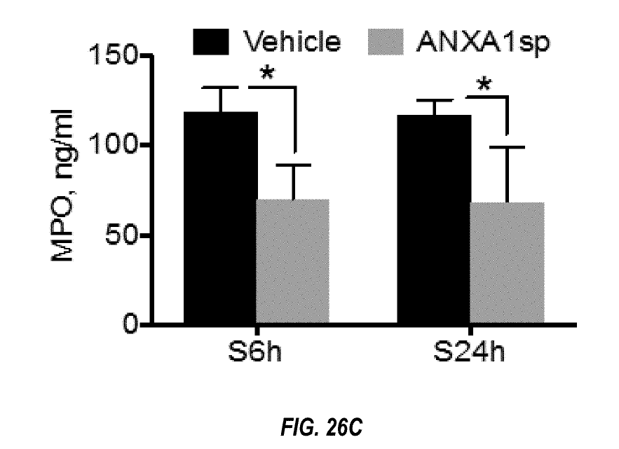

Systemic cytokines, including IL-6, are found elevated in patients developing delirium and POCD and may serve as potential biomarkers. Mice after orthopedic surgery show a sustained increase in systemic IL-6, which was significantly reduced by ANXA1sp (FIG. 26A). By 24 hr systemic levels of MPO were also significantly up-regulated after surgery (FIG. 26B) but not in mice treated with ANXA1sp (FIG. 26C). N=4-5 (adult), N=1-5 (aged); **P<0.01. Two-way ANOVA with Multiple Comparisons.

Any patents or publications mentioned in this specification are indicative of the levels of those skilled in the art to which the present disclosure pertains. These patents and publications are herein incorporated by reference to the same extent as if each individual publication was specifically and individually indicated to be incorporated by reference. In case of conflict, the present specification, including definitions, will control.

One skilled in the art will readily appreciate that the present disclosure is well adapted to carry out the objects and obtain the ends and advantages mentioned, as well as those inherent therein. The present disclosures described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

TABLE 1
Properties and functions of mammalian sirtuins

| | Molecular | | | Key regulatory |
|---|---|---|---|---|
| Sirtuin | mass | Cellular localization | Activity | functions |
| SIRT1 | 81.7 kDa | Nucleus and | Deacetylase | Metabolism, |
| | | cytosol | | inflammation |
| SIRT2 | 43.2 kDa | Cytosol | Deacetylase | Cell cycle and motility, |
| | | | | myelination |
| SIRT3 | 43.6 kDa | Mitochondria | Deacetylase | Fatty acid oxidation, |
| | | | | antioxidant defences |
| SIRT4 | 35.2 kDa | Mitochondria | ADP-ribosyl- | Amino acid-stimulated |
| | | | transferase | insulin secretion, |
| | | | | suppression of fatty |
| | | | | acid oxidation |
| SIRT5 | 33.9 kDa | Mitochondria | Deacetylase? | Urea cycle |
| | | | Demalonylase | |
| | | | Desuccinylase | |
| SIRT6 | 39.1 kDa | Nucleus | Deacetylase | Genome stability, |
| | | | ADP-ribosyl- | metabolism |
| | | | transferase | |
| SIRT7 | 44.8 kDa | Nucleolus | Deacetylase? | Ribosomal DNA |
| | | | | transcription |

That which is claimed is:

1. A method of treating or reducing cognitive decline in a subject following an inflammatory trigger, comprising administering to the subject a therapeutically effective amount of an ANXA1 peptide.

2. A method of ameliorating cognitive decline in a subject following an inflammatory trigger, comprising administering to the subject a therapeutically effective amount of an ANXA1 peptide.

3. A method of preventing cognitive decline in a subject following an inflammatory trigger, comprising administering to the subject a therapeutically effective amount of an ANXA1 peptide.

4. The method of claim 1, wherein the inflammatory trigger comprises a planned inflammatory trigger.

5. The method of claim 4, wherein the planned inflammatory trigger comprises a surgical or medical procedure.

6. The method of claim 1, wherein the cognitive decline comprises post-operative cognitive dysfunction (POCD).

7. The method of claim 1, wherein the ANXA1 peptide comprises ANXA1 short peptide (ANXA1sp): Ac-Gln-Ala-Trp.


SEQUENCE LISTING (9 sequences)

SEQ ID NO: 1 - protein, 1399 aa, Artificial
  peptide
AGTGTGAAATCTTCAGAGAAGAATTTCTCTTTAGTTCTTTGCAAGAAGGTAGAGATAAAGACACTTTTTCAAAAATGGCAATGGTATCAGAATTCCTCAAGCAGGCCTGGTTTATTGAAAATGAAGAGCAGGAATATGTTCAAACTGTGAAGTCATCCAAAGGTGGTCCCGGATCAGCGGTGAGCCCCTATCCTACCTTCAATCCATCCTCGGATGTCGCTGCCTTGCATAAGGCCATAATGGTTAAAGGTGTGGATGAAGCAACCATCATTGACATTCTAACTAAGCGAAACAATGCACAGCGTCAACAGATCAAAGCAGCATATCTCCAGGAAACAGGAAAGCCCCTGGATGAAACACTTAAGAAAGCCCTTACAGGTCACCTTGAGGAGGTTGTTTTAGCTCTGCTAAAAACTCCAGCGCAATTTGATGCTGATGAACTTCGTGCTGCCATGAAGGGCCTTGGAACTGATGAAGATACTCTAATTGAGATTTTGGCATCAAGAACTAACAAAGAAATCAGAGACATTAACAGGGTCTACAGAGAGGAACTGAAGAGAGATCTGGCCAAAGACATAACCTCAGACACATCTGGAGATTTTCGGAACGCTTTGCTTTCTCTTGCTAAGGGTGACCGATCTGAGGACTTTGGTGTGAATGAAGACTTGGCTGATTCAGATGCCAGGGCCTTGTATGAAGCAGGAGAAAGGAGAAAGGGGACAGACGTAAACGTGTTCAATACCATCCTTACCACCAGAAGCTATCCACAACTTCGCAGAGTGTTTCAGAAATACACCAAGTACAGTAAGCATGACATGAACAAAGTTCTGGACCTGGAGTTGAAAGGTGACATTGAGAAATGCCTCACAGCTATCGTGAAGTGCGCCACAAGCAAACCAGCTTTCTTTGCAGAGAAGCTTCATCAAGCCATGAAAGGTGTTGGAACTCGCCATAAGGCATTGATCAGGATTATGGTTTCCCGTTCTGAAATTGACATGAATGATATCAAAGCATTCTATCAGAAGATGTATGGTATCTCCCTTTGCCAAGCCATCCTGGATGAAACCAAAGGAGATTATGAGAAAATCCTGGTGGCTCTTTGTGGAGGAAACTAAACATTCCCTTGATGGTCTCAAGCTATGATCAGAAGACTTTAATTATATATTTTCATCCTATAAGCTTAAATAGGAAAGTTTCTTCAACAGGATTACAGTGTAGCTACCTACATGCTGAAAAATATAGCCTTTAAATCATTTTTATATTATAACTCTGTATAATAGAGATAAGTCCATTTTTTAAAAATGTTTTCCCCAAACCATAAAACCCTATACAAGTTGTTCTAGTAACAATACATGAGAAAGATGTCTATGTAGCTGAAAATAAAATGACGTCACAAGAC

SEQ ID NO: 2 - protein, 346 aa, Artificial
  peptide
MAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALTGHLEEVVLALLKTPAQFDADELRAAMKGLGTDEDTLIEILASRTNKEIRDINRVYREELKRDLAKDITSDTSGDFRNALLSLAKGDRSEDFGVNEDLADSDARALYEAGERRKGTDVNVFNTILTTRSYPQLRRVFQKYTKYSKHDMNKVLDLELKGDIEKCLTAIVKCATSKPAFFAEKLHQAMKGVGTRHKALIRIMVSRSEIDMNDIKAFYQKMYGISLCQAILDETKGDYEKILVALCGGN

SEQ ID NO: 3 - protein, 4 aa, Artificial
  peptide
KQAW

SEQ ID NO: 4 - protein, 4 aa, Artificial
  peptide
FQAW

SEQ ID NO: 5 - protein, 6 aa, Artificial
  peptide
FLKQAW

SEQ ID NO: 6 - protein, 7 aa, Artificial
  peptide
EFLKQAW

SEQ ID NO: 7 - protein, 11 aa, Artificial
  peptide
AMVSEFLKQAW

SEQ ID NO: 8 - protein, 9 aa, Artificial
  peptide
VSEFLKQAW

SEQ ID NO: 9 - protein, 18 aa, Artificial
  peptide
AMVSEFLKQAWFIENEEQ